(19) United States
(12) Patent Application Publication
(10) Pub. No.: US 2023/0235413 A1
(43) Pub. Date: Jul. 27, 2023

(54) A CELLULAR MARKER OF COVID SEVERITY

(71) Applicants: Universite Paris Cite, Paris (FR); Assistance Publique - Hopitaux de Paris, Paris (FR); Centre National de la Recherche Scientifique, Paris (FR); INSERM (Institut National de la Sante et de la Recherche Medicale), Paris (FR); Sorbonne Universite, Paris (FR)

(72) Inventors: Helene Pere, Paris (FR); David Veyer, Paris (FR); Valerie Taly, Bourg-la-Reine (FR); Pierre Laurent-Puig, Meudon (FR)

(21) Appl. No.: 18/009,731

(22) PCT Filed: Jun. 11, 2021
(86) PCT No.: PCT/EP2021/065863

§ 371 Date: Dec. 11, 2022

(30) Foreign Application Priority Data
Jun. 12, 2020 (EP) 20305647.8

(51) Int. Cl. C12Q 1/70; C12Q 1/6883; C12Q 1/686; C12Q 1/06

(52) U.S. Cl. CPC C12Q 1/70; C12Q 1/6883; C12Q 1/686; C12Q 1/06; C12Q 2600/156

(57) ABSTRACT

The invention relates to methods for determining the severity of a disease caused by a coronavirus infection, comprising quantifying the level of cellular RNA of RNase P in a blood sample of a subject.

The invention relates to methods for determining the severity of a disease caused by a coronavirus infection, comprising quantifying the level of cellular RNA of RNase P in a blood sample of a subject.

### TECHNICAL BACKGROUND

Three coronaviruses have crossed the species barrier to cause deadly pneumonia in humans since the beginning of the 21st century: severe acute respiratory syndrome coronavirus (SARS-CoV), Middle-East respiratory syndrome coronavirus (MERS-CoV), and SARS-CoV-2. SARS-CoV-2, was discovered in December 2019 in Wuhan, Hubei province of China. On Jan. 30, 2020, the World Health Organization declared the SARS-CoV-2 epidemic a public health emergency of international concern.

SARS-CoV-2, responsible for the development of COVID-19, was first isolated and sequenced in early January 2020 (Chen Y 2020; Chen L 2020). Although most patients present mild-to-moderate disease, 5 to 10% progress to severe or critical disease (Huang C 2020), including pneumonia and acute respiratory failure. Based on data from patients with laboratory-confirmed COVID-19 from mainland China, admission to intensive care unit (ICU), invasive mechanical ventilation or death occurred in 5.0%, 2.3% and 1.4% of cases, respectively (Guan W-J 2020). In severe cases, clinical observations typically describe a two-step disease progression, starting with a mild-to-moderate presentation followed by a secondary respiratory worsening 9 to 12 days after onset of first symptoms (Grasselli G 2020; Huang C 2020; Li Q 2020). At the time of pulmonary deterioration, although the clinical manifestations of SARS-CoV-2 are dominated by respiratory symptoms, patients may suffer severe systemic damage such as cardiovascular, renal injury and liver injury and/or multiple organ failure (Huang C 2020; Yang F 2020). In addition, coagulopathy has been reported in severe COVID-19 cases (Han 2020; Tang 2020).

The semi-quantitative detection of circulating SARS-CoV-2 RNA as assessed by conventional real-time reverse transcription-polymerase chain reaction assay (RT-PCR) used for routine SARS-CoV-2 molecular diagnosis from respiratory tract samples was previously reported in COVID-19 patients in few studies (Chen X 2020; Chen W 2020; Huang C 2020). The same RT-PCR technology was used to detect blood SARS-CoV-2 RNA in a retrospective limited series of 6 COVID-19 patients (Chen W 2020).

### SUMMARY OF THE INVENTION

While the inventors were attempting to measure SARS-CoV-2 RNA in blood of patients, they used the RNA of RNase P, that is a cellular housekeeping gene, as an internal control to measure. They were extremely surprised to find that the RNA of RNase P was dramatically increasing, reflecting the severity of the disease.

The inventors have more particularly shown that the level of circulating RNA of RNase P increased with the severity of COVID-19.

On that basis, it is provided a method for classifying a subject infected with SARS coronavirus, which method comprises measuring the quantity of circulating RNA of RNase P in a sample of the subject, wherein the sample is a blood, plasma or serum sample of the subject.

The method of the invention is particularly useful to identify subjects with a severe and critical status at an early stage.

It is also provided a method for determining a risk for a subject to develop or show an aggravation of, a SARS coronavirus-induced acute pulmonary failure and/or systemic damage, which method comprises measuring the quantity of circulating RNA of RNase P in a sample of the subject, wherein the sample is a blood, plasma or serum sample of the subject.

It is further provided a method for monitoring efficacy of a therapeutic treatment against a SARS coronavirus infection or SARS coronavirus-induced acute pulmonary failure and/or systemic damage in a subject, which method comprises measuring the quantity of circulating RNA of RNase P in a sample of the subject, wherein the sample is a blood, plasma or serum sample of the subject collected at different points of time before, and during and/or after the subject has been subjected to the therapeutic treatment.

The above methods are performed in vitro.

### LEGENDS TO THE FIGURE

FIGS. 1A et 1B show plasmatic RNA of RNase P (cp/mL) measured by droplet-based digital PCR according to clinical classes.

FIG. 2 shows plasmatic RNA of RNase P (cp/mL) measured by droplet-based digital PCR according to the mechanical ventilation status.

FIG. 3 shows the survival probability for patients with a plasmatic RNase P RNA concentration above 4.63 log cp/mL (the median value of the log plasmatic RNase P RNA concentration in the dataset).

### DETAILED DESCRIPTION OF THE INVENTION

The inventors have provided significant evidence that support quantification of plasmatic RNase P RNA concentration as a prognostic tool for the early detection and monitoring of cell and tissue injury associated with COVID-19. Ordinarily, circulating endogenous RNA is considered to be too fragile and not sufficiently stable to represent a marker to monitor compared to circulating DNA.

### Definitions

The “subject” or “patient” to be treated may be any mammal, preferably a human being. The human subject may be a child, an adult or an elder. The subject has been infected with a SARS coronavirus. In a preferred embodiment, the subject has been diagnosed with COVID-19 and/or have been tested positive with a SARS-CoV-2 RT-PCR testing on a respiratory sample (e.g. a nasopharyngeal swab or an invasive respiratory sample).

The term “infection by a SARS coronavirus” includes any stage of infection. In particular embodiments, the coronavirus may be SARS-CoV, and SARS-CoV-2, preferably SARS-CoV-2, responsible for the COVID-19 pandemic.

In particular embodiments, the coronavirus may one or several of SARS-CoV-2 lineages or SARS-CoV-2 variants such as: Lineage B or Wuhan
Lineage B.1
Lineage B.1.1.7 or British variant
Lineage B.1.351 or South African variant
Lineage B.1.1.207 or Nigerian variant,
Lineage B.1.1.248 or Japan or Brazil variant
Lineage B.1.617 or Indian variant

The term “RNAaemia” means the level of RNA in a blood, serum, or plasma sample. The present invention is concerned with the RNAaemia of cellular RNA of RNase P, which should not be confused with the viral RNAaemia. In the present disclosure, the term “RNAaemia” when referring to the cellular RNA of RNase P, refers to the level (in term of quantity or concentration) of cellular RNA of RNase P in a blood, serum, or plasma sample.

As used herein, the term “treatment” or “therapy” includes curative and/or prophylactic treatment. More particularly, curative treatment refers to any of the alleviation, amelioration and/or elimination, reduction and/or stabilization (e.g., failure to progress to more advanced stages) of a symptom, as well as delay in progression of a symptom of a particular disorder. In the context of the present invention, a curative treatment more particularly refers to reducing the risk of worsening of the disease, especially COVID-19. Particularly, the treatment may aim at preventing a mild or moderate stage ARDS from developing to a more severe stage (in reference to the Berlin definition, defined below), and ultimately at preventing death of the patient. Prophylactic treatment refers to any of: halting the onset, reducing the risk of development of disease, reducing the incidence, delaying the onset, reducing the development as well as increasing the time to onset of symptoms of a particular disorder. In the context of the present invention, the prophylactic treatment more particularly refers to preventing or minoring symptoms of the infection by the coronavirus, in particular preventing the disease, especially COVID-19, to develop and to trigger an ARDS.

A patient is diagnosed with a hypoxemia typically when his/her PaO₂/FiO₂ ratio is at most 300 mmHg for a positive tele-expiratory pressure set at minus 5cmH₂O (PaO₂ represents the arterial partial pressure of dioxygen and FiO₂ is the inspired fraction of dioxygen in the gas inhaled by the patient).

An acute respiratory distress syndrome (ARDS) is a condition that results from an attack on the alveolo-capillary membrane leading to so-called “lesional” pulmonary edema. The 2012 Berlin conference (Ranieri et al. 2012), defined ARDS by 4 criteria: 1) an onset of acute respiratory symptoms within 7 days of an alveolar attack, 2) hypoxemia which results in a PaO₂/FiO₂ ratio ≤300 mmHg for a positive tele-expiratory pressure set at minus 5cmH₂O, 3) the presence of bilateral pulmonary opacities in chest imaging, 4) pulmonary edema which is not explained by the preferential increase in hydrostatic pressure. The ARDS case fatality rate is inversely associated with the value of the PaO₂/FiO₂ ratio.

TABLE 1
Berlin Definition of ARDS. Ranieri et al, 2012

| | |
|---|---|
| Timing | Within 1 week of a known clinical insult or new or |
| | worsening respiratory symptoms |
| Chest | Bilateral opacities- not fully explained by effusions, |
| imaging^{a} | lobar/lung collapse, or nodules |
| Origin of | Respiratory failure not fully explained by cardiac |
| edema | failure or fluid overload |
| | Need objective assessment (e.g. echocardiography) |
| | to exclude hydrostatic edema if no risk factor present |
| Oxygenation^{b} | 200 mm Hg < PaO₂/FiO₂ ≤ 300 mm Hg with PEEP or |
| MILD | CPAP ≥ 5 cm H₂O^{c} |
| MODERATE | 100 mm Hg < PaO₂/FiO₂ ≤ 200 mm Hg with PEEP or |
| | CPAP ≥ 5 cm H₂O |
| SEVERE | PaO₂/FiO₂ ≤ 100 mm Hg with PEEP or CPAP ≥ 5 |
| | cm H₂O |

Abbreviations CPAP, continuous positive airway pressure; FiO₂, fraction of inspired oxygen; PaO₂, partial pression of arterial oxygen; PEEP, positive end-expiratory pressure.
^{a}chest radiograph or computed tomography scan.
^{b}if altitude is higher than 1000 m, the correction factor should be calculated as follows [PaO₂/FiO₂ × (barometric pressure/760)].
^{c}this may be delivered noninvasively in the mild acute respiratory distress syndrome group.

As used herein, the term “amplification” refers to a process that increases the representation of a population of specific nucleic acid sequences in a sample by producing multiple (i.e., at least 2) copies of the desired sequences. Methods for nucleic acid amplification are known in the art and include, but are not limited to, polymerase chain reaction (PCR). A “copy” or “amplicon” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable but not complementary to the template), and/or sequence errors that occur during amplification. A typical amplification reaction is carried out by contacting a forward and reverse primer (a primer pair) to the sample DNA together with any additional amplification reaction reagents under conditions which allow amplification of the target sequence.

### RNA Extraction and Amplification

A sample of blood is obtained from a subject according to methods well known in the art. In the method of the invention, the RNA may be measured from a whole blood sample. In another preferred embodiment, the method makes use of plasma or serum samples.

Plasma or serum may be isolated according to methods known in the art.

As an example, RNA may be extracted from the blood, plasma or the serum immediately or within 1 hour, 2 hours, 3 hours, 4 hours, 5 hours or 6 hours. Optionally the plasma or serum can be stored after blood centrifugation at −20° C. or −80° c. prior to isolation of the RNA.

Methods of RNA extraction are well known in the art.

In a particular embodiment, the RNA from the sample may be fractionated prior to performing an amplification reaction. In a preferred embodiment, the amplification reaction is a digital droplet PCR reaction (ddPCR).

It is thus herein described a method for quantifying circulating RNA of RNase P in a sample of the subject, wherein the sample is a blood, plasma or serum sample of the subject, which method comprises extracting the RNA from the sample and amplifying the RNA by a reverse transcriptase digital droplet PCR.

Droplet-based digital PCR is based on the realization of thousands to millions of single-molecule PCRs in parallel in independent compartments and the resulting amplification products reflects more closely the original composition of nucleic acid mixtures than conventional PCR while in parallel being more tolerant to presence of inhibitors compared to bulk-based systems (Pekin D 2011; Perkins G 2017; Taly V 2012; Yu F 2020).

Digital PCR samples are partitioned into thousands of independent endpoint PCR reactions prior to amplification, and a reaction well is scored as either positive or negative for amplification of the viral sequence of interest. The positive wells are counted and converted to a concentration of target in the original sample. This binary assignment of each reaction greatly minimizes the measurement's dependency on parameters such as assay efficiency and instrument calibration. As a result, this enables different laboratories to compare viral load measurement results in a standardized manner without interference from external factors.

A description of this technology is provided e.g. in international patent application WO2015/013681, incorporated herein by reference.

To fractionate the RNA sample/specimen, emulsification techniques can be used so as to create large numbers of aqueous droplets that function as independent reaction chambers for the PCR reactions. For example, an aqueous specimen (e.g., 20 microliters) can be partitioned into droplets (e.g., 20,000 droplets of one nanoliter each) to allow an individual test for the target to be performed within each of the droplets.

Aqueous droplets can be suspended in oil to create a water-in-oil emulsion (W/O). The emulsion can be stabilized with a surfactant to reduce coalescence of droplets during heating, cooling, and transport, thereby enabling thermal cycling to be performed.

In an exemplary droplet-based digital assay, a specimen is partitioned into a set of droplets at a dilution that ensures that more than 40 percent, preferably more than 50, 60, 70, 80, or 90 percent of the droplets contain no more than one RNA molecule per specimen fraction.

Once fractionation has taken place, the RNA may then optionally be amplified.

The primers and probes used for amplification need not reflect the exact sequence of the target nucleic acid sequence (i.e. need not be fully complementary), but must be sufficiently complementary so as to hybridize to the target site under the particular experimental conditions. Accordingly, the sequence of the oligonucleotide typically has at least 70 percent homology, preferably at least 80 percent, 90 percent, 95 percent, 97 percent, 99 percent or 100 percent homology, for example over a region of at least 13 or more contiguous nucleotides with the target sequence. The conditions are selected such that hybridization of the oligonucleotide to the target site is favored and hybridization to the non-target site is minimized.

In certain embodiments, the detection probes or amplification primers or both probes and primers are labeled with a detectable agent or moiety before being used in amplification/detection assays. In certain embodiments, the detection probes are labeled with a detectable agent. Preferably, a detectable agent is selected such that it generates a signal which can be measured and whose intensity is related (e.g., proportional) to the amount of amplification products in the sample being analyzed.

### Target Sequence to Quantify: RNA of RNase P

Ribonuclease P (RNase P) is an ubiquitous intracellular endoribonuclease that cleaves other RNA molecules at the junction between a single-stranded region and the 5′ end of a double-stranded region. The enzyme is made up of two subunits. The RNA subunit, itself, is catalytic. The present methods measure the quantity of the RNA subunit. In humans, the RNA subunit of RNase P is also named H1 RNA (the sequence of which is disclosed e.g. in GENEBANK Access No. NR_002312).

In a preferred embodiment, the methods of the invention comprise determining the RNase P RNAaemia in a human patient by selectively amplifying said RNA obtained from a blood, serum or plasma sample.

Correlation with Clinical Severity and Prediction of Aggravation

The method described herein allows to classify a subject infected with SARS coronavirus, according to the severity of the infection.

The mechanism underlying the correlation between the RNase P RNAaemia and the severity of the disease remains unknown.

The method described herein further allows to determine the risk for a subject to develop, or show an aggravation of, a SARS coronavirus-induced acute pulmonary failure and/or systemic damage.

Such system damage includes e.g. cardiovascular injury, renal injury, liver injury and/or multiple organ failure.

All can lead to the death of the subject.

The higher the RNAaemia of the RNase P, the higher the severity of the condition and the risk for the subject. According to the invention, the quantification of circulating RNA of the RNAase P is thus a marker of severity, as well as a marker for predicting the clinical development and outcome, or a marker for determining the risk for the subject to develop symptoms, or to show an aggravation of SARS coronavirus-induced acute pulmonary failure and/or systemic damage, and risk of death.

In a preferred embodiment, especially useful when the SARS coronavirus is SARS-CoV-2, the sample is collected between 5 to 15 days, preferably between 8 to 12 days, still preferably about 10 days, after first symptoms of the SARS-induced disease (respectively COVID-19), including e.g. fever and/or dry cough.

In another preferred embodiment, also especially useful when the SARS coronavirus is SARS-CoV-2, the sample is collected at DO and/or at different points of time between DO and D10, preferably at DO, D3, D6, D9, wherein
D0 (day 0) is the first day of symptoms, including e.g. fever and/or dry cough, and D10 is the tenth day after at least a first symptom has occurred, or
D0 (day 0) is the day wherein the subject has been diagnosed with a SARS virus infection, preferably wherein the subject has been tested positive with a SARS virus (e.g. SARS-CoV-2) RT-PCR testing on a respiratory sample (e.g. a nasopharyngeal swab or an invasive respiratory sample).

According to the method of the invention, an RNAaemia of RNase P that is 30 000 or more copies/mL (about 4.5 log₁₀) at D0 is indicative of a subject that is a severe case and should be closely monitored.

Patients can be typically classified as follows (based on an adaptation of the Sixth Revised Trial Version of the Novel Coronavirus Pneumonia Diagnosis and Treatment Guidance)

Mild cases: The clinical symptoms (e.g. fever, myalgia, fatigue, and/or diarrhea) are mild, and there is no sign of pneumonia on imaging, e.g. on thoracic computed tomography (CT) scan.

Moderate cases: Showing fever and respiratory symptoms (such as dyspnea) with radiological findings of pneumonia, e.g. on thoracic CT scan. Such patients generally require a maximum of 3 L/min of oxygen.

Severe cases: Cases meeting any of the following criteria: i) Respiratory distress (≥30 breaths/min); ii) Oxygen saturation≤93% at rest; iii) Arterial partial pressure of oxygen (PaO2)/fraction of inspired oxygen (FiO2)≤300 mmHg (1 mmHg=0.133 kPa). PaO2/FiO2 in high-altitude areas (at an altitude of over 1,000 meters above the sea level) shall be corrected by the following formula: PaO2/FiO2×[Atmospheric pressure (mmHg)/760] Cases with chest imaging that showed obvious lesion progression within 24-48 hours >50% are managed as severe cases. Such patients generally require at least 3 L/min of oxygen with no other organ failure.

Critical cases: Cases meeting any of the following criteria: i) Respiratory failure and requiring mechanical ventilation; ii) Shock; and/or iii) With other organ failure that requires intensive care.

An RNAaemia of RNase P below to 3.5 log₁₀ copies (cp)/mL (namely below 3000 copies/ml), preferably between 0 and 1000 cp/ml, is typically indicative of a mild case.

An RNAaemia of RNase P that is between 4 and 4.5 log₁₀ copies (cp)/mL (namely between about 10,000 and 30,000 copies/ml), is typically indicative of a moderate to severe case.

Furthermore an RNAaemia of RNase P equal or superior to 4.5 log₁₀ cp/mL, e.g. equal or superior to about 30,000 cp/ml, is indicative of a severe to critical case.

More specifically, an RNAaemia of RNase P equal or superior to 5 log₁₀ cp/mL is indicative of a critical case.

In a preferred embodiment, the subject shows hypoxemia.

In a particular aspect the subject shows hypoxemia and the method is for use in assessing the risk of aggravation to a more severe stage of pulmonary failure and/or systemic damage, or death.

The method also allows to determine whether patients with mild or moderate symptoms, including e.g. dyspnea, shortness of breath and respiratory distress, including any or several symptoms such as polypnea, cyanosis, grunting, nose flaring, sweating, wheezing, and/or chest retractions, are likely to develop aggravated symptoms, which would require an emergency care to avoid or delay developing into a severe or critical case. The method further allows to determine whether patients with severe symptoms are likely to worsen.

In still a particular embodiment, the subject has an ARDS, preferably a mild or moderate ARDS, and the method allows to determine the risk for the subject to show a severe ARDS. In a particular aspect, an RNAaemia of RNase P of 4.5 log₁₀ copies/mL or more, e.g. more than 4.6 log₁₀ copies/mL, is indicative of a subject likely develop a deterioration during his follow-up as acute pulmonary failure event, such as an acute respiratory distress syndrome (ARDS), and/or systemic damage, e.g. cardiovascular injury, renal injury, liver injury and/or multiple organ failure or death.

In a special embodiment, it is provided a method for determining whether a subject infected with SARS coronavirus, preferably SARS-CoV-2, is likely to die of the infection, which method comprises measuring the quantity of circulating RNA of RNase P in a sample of the subject, wherein the sample is a blood, plasma or serum sample of the subject, and a quantity of circulating RNA of RNase P of 4.5 log₁₀ copies/mL or more is indicative that the subject is at high risk of dying within three weeks.

The term “high risk” typically refers to a multiplication of the risk by at least 2, at least 3, or preferably at least 4, at least 5, or at least 6, compared to a subject which shows an lower level of circulating RNA of RNase P.

In a preferred embodiment, the sample has been collected at D0 and/or at any day between D0 and D10, wherein D0 (day 0) is the first day of symptoms of a SARS-induced disease or is the day wherein the subject has been diagnosed with a SARS coronavirus infection, wherein a quantity of circulating RNA of RNase P of 4.5 log₁₀ copies/mL or more is indicative that the subject is at high risk of dying within one to three weeks from DO.

### Monitoring of Treatment

The method described herein further allows monitoring efficacy of a therapeutic treatment against a SARS coronavirus infection or SARS coronavirus-induced acute pulmonary failure and/or systemic damage in a subject.

For that purpose, samples are collected in a patient who undergoes a candidate therapeutic treatment, at different points of time, preferably at least one time before initiation of the therapeutic treatment, and at least another time during the course of the treatment, or after the treatment. Assessing the sensitivity of a subject with respect to a candidate treatment makes it possible to adjust the dosage or regimen of the treatment or to change treatment.

In a preferred embodiment, the subject is at an early stage of the disease, e.g. they show early symptoms such as fever and/or dry cough.

The method more particularly allows to determine whether a patient with mild or moderate symptoms, including dyspnea, shortness of breath and respiratory distress, including any or several symptoms such as polypnea, cyanosis, grunting, nose flaring, sweating, wheezing, chest retractions, benefits from the candidate treatment.

Examples of candidate therapeutic treatments include antimicrobial agents such as antifungals or antibiotics, antiviral agents and/or immunomodulators.

Anti-microbial therapeutic agents of interest encompass, without being limited to, remdesevir, lopinavir, ritonavir, favipiravir, camostat mesylate, azithromycin, chloroquine, hydroxy-chloroquine.

Immunomodulators encompass, without being limited to, immunosuppressants such as cyclosporine, mycophenolate, anti-IL6, anti-IL1, interferons, and/or biotherapies.

A stagnation or a decrease of the RNAaemia of RNase P, preferably by at least 10%, 20%, or 30%, upon treatment indicates that the therapeutic treatment is effective in the subject. The stronger decrease, the better the prognosis for the subject.

An increase of the RNAaemia of RNase P is indicative that the treatment is ineffective and of a bad prognosis. Such cases compel a change of treatment and an intensive care of the subject. In another aspect it is herein described a method for treating a subject infected with a SARS coronavirus, especially for treating COVID-19, which method comprises quantifying the RNAaemia of RNase P, so as to assess the status of the subject and the risk of aggravation, and administering a therapeutic treatment to the subject, to fight against development and/or worsening of acute pulmonary failure, such as an acute respiratory distress syndrome (ARDS), and/or systemic damage, e.g. cardiovascular injury, renal injury, liver injury and/or multiple organ failure and/or death.

The following examples illustrate the invention without limiting its scope.

### Example 1: Quantification of Circulating RNase P (RNAaemia) by Droplet-Based Digital PCR

### Methods

### Study Design and Patients

Patients admitted to Hôpital Cochin, Paris, France, at the time of respiratory deterioration and between days 8 and 12, were included between Mar. 19, 2020 and Apr. 3, 2020, in the setting of the local RADIPEM biological samples collection derived from samples collected in routine care. Biological collection and informed consent were approved by the Direction de la Recherche Clinique et Innovation (DRCI) and the French Ministry of Research (N^{o} 2019-3677). Inclusion criteria for COVID-19 inpatients were: age between 18 and 80 years old, diagnosis of COVID-19 according to WHO interim guidance, and positive SARS-CoV-2 RT-PCR testing on a respiratory sample (nasopharyngeal swab or invasive respiratory sample). In patients with pre-existing unstable chronic disorders (such as uncontrolled diabetes mellitus, severe obesity defined as body mass index greater than 30, unstable chronic respiratory disease or chronic heart disease) were excluded. Since median duration from onset of symptoms to respiratory failure was shown to be 9.5 (interquartile range, 7.0-12.5) days in our cohort, we analyzed SARS-CoV-2 RNAaemia, and RNase P RNAaemia, on samples collected between 8 to 12 days after first symptoms.

Healthy controls were RT-PCR SARS-CoV-2 negative asymptomatic adults, matched with cases on age and sex.

The clinical severity of COVID-19 described according to the adaptation of the Sixth Revised Trial Version of the Novel Coronavirus Pneumonia Diagnosis and Treatment Guidance published on Feb. 19, 2020. Mild cases were defined as patients with mild clinical symptoms (fever, myalgia, fatigue, and diarrhea) and no sign of pneumonia on thoracic computed tomography (CT) scan. Moderate cases were defined as patients with clinical symptoms associated with dyspnea and radiological findings of pneumonia on thoracic CT scan, and requiring a maximum of 3 L/min of oxygen. Severe cases were defined as respiratory distress patients requiring aver 3 L/min of oxygen with no other organ failure. Critical cases were defined as patients requiring mechanical ventilation, into shock and/or with other organ failures that required intensive care unit (ICU) cares. The study conforms to the principles outlined in the Declaration of Helsinki, and received approval by the appropriate Institutional Review Board (Cochin-Port Royal Hospital, Paris, France; number AAA-2020-08018).

### Quantification of RNAaemia of SARS-CoV-2 and RNAaemia of RNaseP

Plasma RNA (140 μL) was extracted using QIAamp® Viral RNA Mini Kit (QIAGEN®, Hilden, Germany), according to manufacturer's instructions. SARS-CoV-2 RNAaemia was quantified at each time point by droplet-based Crystal Digital PCR™ (Stilla Technologies, Villejuif, France) on the Naica™ System (Stilla Technologies, Villejuif, France), which includes primers and FAM- and HEX-labeled probes specific to two distinct regions [ORF1ab and Nucleocapside (N) genes] of the SARS-CoV-2 positive strand RNA genome. The 3^{rd} channel of the Naica™ system was used as an endogenous PCR control detecting a human housekeeping gene RNase P with a Cy5-labeled probe. This single assay design permits the simultaneous detection of two independent SARS-CoV-2 sequences, while concurrently monitoring PCR effectiveness using the third channel of detection. Plasma samples with one of the two ORF1 or N genes or both genes detected were considered as positive samples and results were automatically analyzed using “Crystal reader” (Stilla) and “Crystal Miner” software (Stilla) based on the most amplified gene positive droplets. SARS-CoV-2 RNA and RNase P concentrations (cp/mL) were finally calculated considering the extracted volume of plasma.

### Statistical Analysis

Descriptive statistics were computed for the population at baseline. Quantitative variables were described as mean±standard deviation (SD) if normally distributed, or median and inter-quartile range (IQR) otherwise. Categorical variables were described as group sizes and percentages.

Bivariate comparisons between clinical classes were computed using the Fisher test for categorical variables and the Kruskal-Wallis one-way analysis of variance for continuous variables. When comparing control and COVID-19 patients, the Mann-Whitney rank-sum test was used for quantitative variables.

An ordinal regression multivariate model was used to evaluate the association between the clinical class and circulating viral DNA quantification (log values), adjusted for clinical predictors. We simply described the patients' clinical outcome using Kaplan-Meier curves and we did not perform statistical survival analysis, as this study was not designed for survival analysis, the patients belonging to different clinical classes at baseline, and the outcome being a composite criterion of mechanical ventilation requirement or death.

Computations were performed using the R software, and the ordinal package for the ordinal regression model. P values <0.05 were considered significant.

### Results

### Patients Characteristics

Fifty-eight COVID-19 patients and 12 healthy controls were analyzed. Demographic and clinical characteristics of the patients are shown in Table 2 below. Median age of the patients was 55.1 years (interquartile range, 15) and 81% were male.

On admission, the degree of severity of COVID-19 was categorized as mild-to-moderate in 17 patients (median oxygen requirement 2 L/min), severe in 16 patients (median oxygen requirement 5 L/min) and critical in 26 patients. All of 55 CT scan available at time of admission were abnormal, showing ground-glass opacities (100%) with bilateral patchy distribution (95%). No patients with mild-to-moderate disease required admission to ICU or use of mechanical ventilation, while 5 out of 15 patients with severe disease were admitted to ICU. No healthy controls and 55% of COVID-19 patients did not present any comorbidity. In the remaining COVID-19 patients (45%), the controlled comorbidities described were non-insulin diabetes mellitus (n=7), solid cancers (n=6), hematological cancer (n=3), asthma (n=3), autoimmune diseases (n=3) and chronic obstructive pulmonary disease (n=1). Fifteen COVID-19 patients received an antiviral treatment at time of their hospitalization for disease progression.

TABLE 2
Clinical characteristics of hospitalized COVID-19 patients

| | N | % | Med | IQR |
|---|---|---|---|---|
| Sexe | 58 | | | |
| Female | 11 | 19 | | |
| Male | 47 | 81 | | |
| Age | 58 | | 55.14 | 15 |
| Clinical classes | 58 | | | |
| Mild/Moderate | 17 | 29 | | |
| Severe | 15 | 26 | | |
| Critical | 26 | 45 | | |
| Clinical Degradation | 58 | | | |
| Death | 3 | 5.2 | | |
| Mechanical Ventilation | 3 | 5.2 | | |
| Optiflow | 3 | 5.2 | | |
| Absence | 49 | 84 | | |
| Monitoring delay | 58 | | 7 | 11 |
| Pulmonary radiography showing ground- | 55 | | | |
| glass opacities | | | | |
| No | 0 | 0 | | |
| Yes | 55 | 100 | | |
| Pulmonary radiography showing bilateral | 55 | | | |
| patchy distribution | | | | |
| No | 3 | 5.5 | | |
| Yes | 52 | 95 | | |
| Controlled comorbidities | 58 | | | |
| No | 32 | 55 | | |
| Yes | 26 | 45 | | |
| Oxygeno-requerance | 58 | | | |
| No | 3 | 5 | | |
| Yes | 29 | 50 | | |
| Mechanical Ventilation | 26 | 45 | | |
| Mechanical Ventilation | 26 | 45 | | |
| Antiviral treatment | 58 | | | |
| No | 43 | 74 | | |
| Yes | 15 | 26 | | |

IQR: Interquartile range

RNase P RNAaemia Results by ddPCR

RNase P RNAaemia correlated with COVID-19 severity (p=10⁻¹⁰), as depicted in the Figure. See also Table 3.

TABLE 3
ddPCR RNase P
ddPCR RNaseP

| Clinical | Mild/Moderate | | | | | Severe | | | Critical | | Stat | |
|---|---|---|---|---|---|---|---|---|---|---|---|---|
| classes | N | % | Median | IQR | N1 | Median1 | IQR1 | N2 | Median2 | IQR2 | p | test |
| human RNA | 17 | | 18929 | 21667 | 15 | 50773.81 | 74345 | 26 | 121309.5221 | 149271 | 2.6e−06 | kruskal.test |
| (cp/mL | | | | | | | | | | | | |
| plasma) | | | | | | | | | | | | |
| log(human | 17 | | 4.3 | 0.45 | 15 | 4.705639754 | 0.56 | 26 | 5.083631182 | 0.58 | 2.6e−06 | kruskal.test |
| RNA) | | | | | | | | | | | | |
| (cp/mL | | | | | | | | | | | | |
| plasma) | | | | | | | | | | | | |

### Example 2: Quantification of Circulating RNase P (RNAaemia) by Droplet-Based Digital PCR in Merged Cohorts

To complete and extend the observations of RNAase P RNA concentration (RNAaemia) quantified by ddPCR as biomarker of COVID-19 clinical severity and outcome, another series of 79 patients hospitalized for COVID-19 was retrospectively included during the first wave of the epidemic in France between March and July 2020. In this global study bringing together two cohorts of comparable COVID-19, the absolute quantification of circulating RNase P (RNAaemia) was reported in the plasma of 139 COVID-19 hospitalized patients. The interest of this marker was evaluated in specifying the degree of clinical severity of COVID-19, and correlated with the clinical outcome by assessing the degree of systemic viral invasion in COVID-19 patients at hospital admission.

### Study Design and Patients

Two cohorts of respectively 60 COVID-19 patients admitted to the Cochin Hospital, Paris, France, and 79 to the European George Pompidou Hospital (HEGP), Paris, France, were included between Mar. 19, 2020 and Jun. 26, 2020. Inclusion criteria for COVID-19 inpatients were: age between 18 and 80 years, diagnosis of COVID-19 according to World Health Organization (WHO) interim guidance, and positive SARS-CoV-2 RT-PCR testing on a respiratory sample (nasopharyngeal swab or invasive respiratory sample). The clinical severity of COVID-19 described according to the adaptation of the Sixth Revised Trial Version of the Novel Coronavirus Pneumonia Diagnosis and Treatment Guidance published on Feb. 19, 2020.

Mild cases, moderate cases, severe cases and critical cases were as defined in Example 1.

RNA extraction, and quantification of plasmatic RNase P were conducted substantially as described in Example 1.

Statistical analysis. Descriptive statistics were computed for the population at baseline. Quantitative variables were described as mean±standard deviation (SD) if normally distributed, or median and inter-quartile range (IQR) otherwise. Categorical variables were described as group sizes and percentages.

Bivariate comparisons between clinical classes were computed using the Fisher test for categorical variables and the one-way analysis of variance for continuous variables when all groups were normally distributed and the Kruskal-Wallis one-way analysis of variance otherwise. When comparing control and COVID-19 patients, the Student t test was used for quantitative variables when both groups were normally distributed, and the Mann-Whitney rank-sum test otherwise.

The Cox proportional hazards model was used to evaluate the risk of death at inclusion between patients with low and high plasmatic RNase P concentration.

Patients' clinical outcomes are presented using Kaplan-Meier curves. We did not perform a formal statistical survival analysis, since the study was not primarily designed for survival analysis and the patients belonging to different clinical classes at baseline.

Computations were performed using the R software, and the survival package for the Cox proportional hazards model. P values <0.05 were considered significant.

### Results

Patient characteristics. One hundred and thirty-nine hospitalized COVID-19 patients were included. Global and per-cohort demographic and clinical characteristics of the patients are shown in Table 4. Patients were 59 years old on average (SD=14) and 78% were male. Patients in both cohorts were globally comparable, although the Cochin Hospital cohort patients were slightly younger (mean age 55 vs 62) and presented less comorbidities (30% vs 53% hypertension, 3% vs 15% chronic renal failure) at inclusion than patients in the HEGP cohort.

At inclusion, the degree of severity of COVID-19 was categorized as mild-to-moderate in 37 (27%) patients, severe in 35 (25%) patients and critical in 67 (48%) patients. The median time from symptom onset to plasma sample was of 11 days in the global merged cohort (respectively 10 days and 13 days for Cochin Hospital and HEGP cohort). Seventy-six (54%) of the COVID-19 patients do not present any comorbidities. In the remaining COVID-19 patients (63, 46%), comorbidities were: diabetes (n=29; 25/29 are isolated non-insulin diabetes mellitus), cardiovascular history (n=21), hypertension (n=60), cancer history (n=13) and chronic renal failure (n=14).

TABLE 4
Demographic and clinical findings of 139 patients
suffering from COVID-19 hospitalized in Paris
during the first wave of the epidemic.

| | N | % | Mean | Med | IQR |
|---|---|---|---|---|---|
| Age | 139 | | 59 | 58.61 | 17.30 |
| Sex | 139 | | | | |
| Women | 31 | 22 | | | |
| Men | 108 | 78 | | | |
| Classes | 139 | | | | |
| Critical | 37 | 27 | | | |
| Moderate | 35 | 25 | | | |
| Severe | 67 | 48 | | | |
| Delay from Symptoms Onset (DSO) | 139 | | | 11 | 5 |
| Tobacco status | 138 | | | | |
| Active smocking | 4 | 2.9 | | | |
| Never smocker | 109 | 79 | | | |
| Weaned smoker | 25 | 18 | | | |
| Cardiovascular anteriority | 135 | | | | |
| No | 114 | 84 | | | |
| Yes | 21 | 16 | | | |
| Hypertension | 139 | | | | |
| No | 79 | 57 | | | |
| Yes | 60 | 43 | | | |
| Diabetes | 139 | | | | |
| No | 110 | 79 | | | |
| Yes | 29 | 21 | | | |
| Cancer anteriority | 139 | | | | |
| No | 126 | 91 | | | |
| Yes | 13 | 9.4 | | | |
| Chronic renal failure | 139 | | | | |
| No | 125 | 90 | | | |
| Yes | 14 | 10 | | | |
| Mechanical ventilation | 67 | 48 | | | |
| Pulmonary severity (% of | 115 | | | | |
| lung involvement) | | | | | |
| <10% | 12 | 10 | | | |
| 10-25% | 22 | 19 | | | |
| 25-50% | 46 | 40 | | | |
| 50-75% | 29 | 25 | | | |
| 75-100% | 6 | 5.2 | | | |
| Death | 139 | | | | |
| No | 112 | 81 | | | |
| Yes | 27 | 19 | | | |

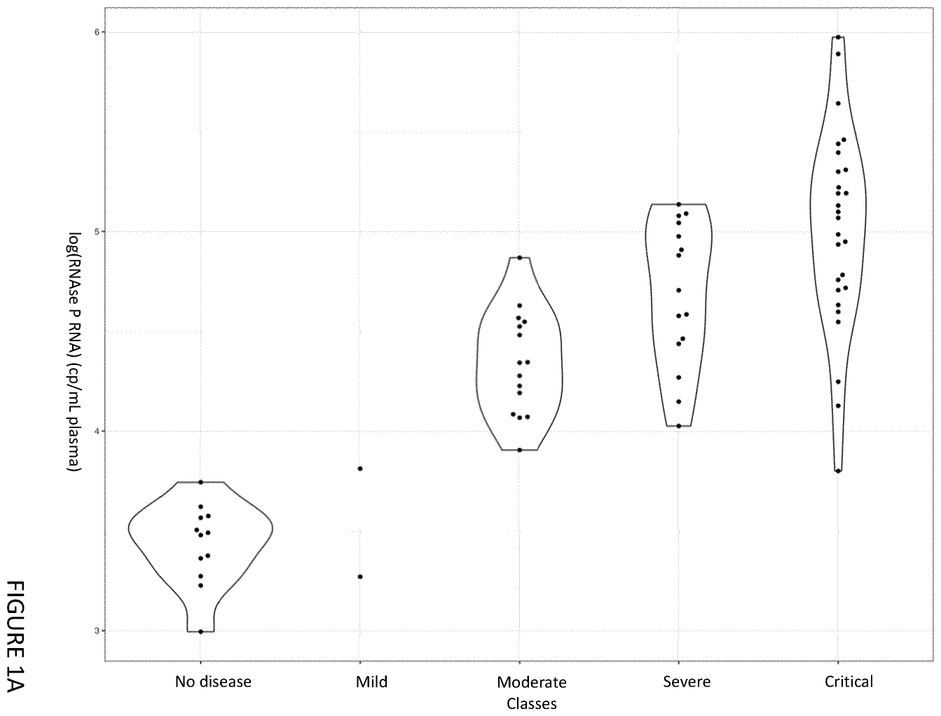

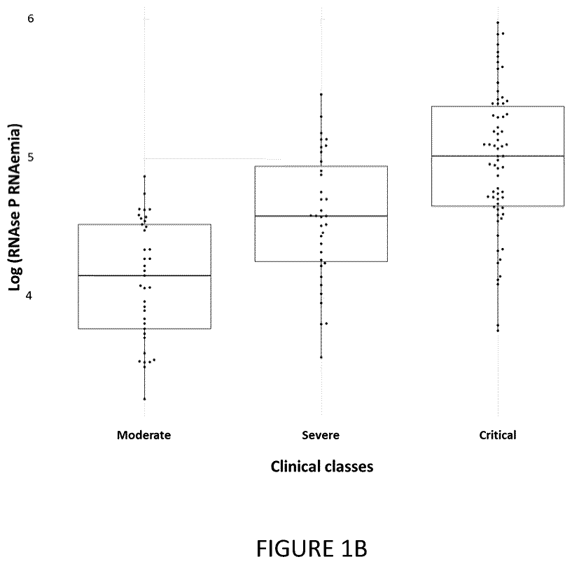

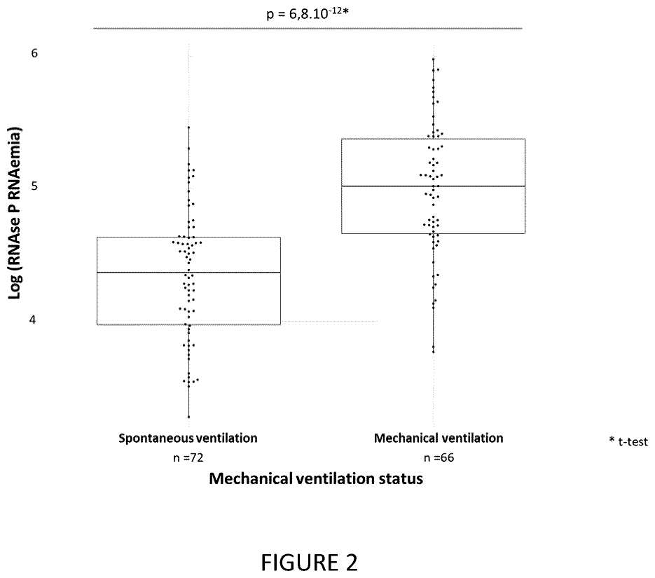

Correlation between plasmatic RNase P concentration determined by ddPCR and clinical classes and intubation status. Plasmatic RNase P concentration was highly and significantly correlated with clinical severity classes (Table 5A; ANOVA p=3.4e-14 on log values; FIG. 1B) and with the mechanical ventilation status (Table 5B; Student t test p=6.8e-12; FIG. 2) with a mean RNase P plasmatic concentration respectively of 4.3 log cp/mL (SD=0.5, median=23155 cp/mL) in non-intubated patients vs Slog cp/mL (SD=0.51, median=103482 cp/mL) in intubated patients.

TABLE 5
Plasmatic RNase P concentrations in 139 patients suffering from COVID-19 according
to clinical severity (A) and to the mechanical ventilation status (B).
A. Correlation with COVID-19 clinical severity

| | Moderate | | | Severe | | | Critical | | | |
|---|---|---|---|---|---|---|---|---|---|---|
| | N | Med | IQR | N | Med | IQR | N | Med | IQR | p |
| RNaseP | 37 | 14345 | 27500 | 35 | 38438 | 69702 | 66 | 103482 | 192500 | 5.1e−12 |
| RNAaemia | | | | | | | | | | |
| (cp/mL plasma) | | | | | | | | | | |
| log(RNaseP | 37 | 4.2 | 0.75 | 35 | 4.6 | 0.68 | 66 | 5 | 0.72 | 3.4e−14* |
| RNAaemia) | | | | | | | | | | |

*ANOVA

B. Correlation with mechanical ventilation status

| | Spontaneous ventilation | | | Mechanical ventilation | | | |
|---|---|---|---|---|---|---|---|
| | N | Med | IQR | N | Med | IQR | p |
| RNaseP | 72 | 23155 | 33363 | 66 | 103482 | 192500 | 8.9e−11 |
| RNAaemia | | | | | | | |
| (cp/mL plasma) | | | | | | | |
| log(RNaseP | 72 | 4.4 | 0.66 | 66 | 5 | 0.72 | 6.8e−12* |
| RNAaemia) | | | | | | | |

*t-test

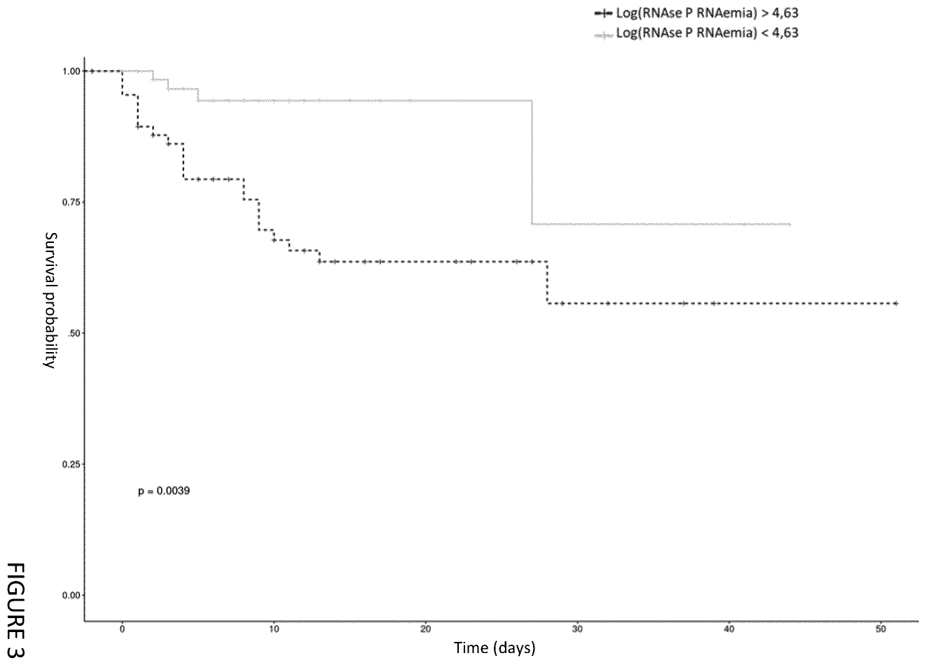

Clinical monitoring and correlation with plasmatic RNase P RNA concentration and intubation status. During hospitalization and clinical monitoring of COVID-19 patients, 27 of the 139 patients died from their infection. Plasmatic RNase P RNA concentration (RNAaemia) at hospital admission allowed to predict overall survival of hospitalized COVID-19 patients. Indeed, patients with a plasmatic RNase P RNA concentration above 4.63 log cp/mL (the median value of the log plasmatic RNase P RNA concentration in the dataset) at admission presented a 4.3 Hazard Ratio (p=0.0039, univariate Cox proportional odds model) to die during their clinical monitoring (FIG. 3).

As a conclusion, the inventors observed that RNase P RNA, an ubiquitous and aspecific human intracellular RNA marker, was highly correlated with disease severity and intubation status in COVID-19 hospitalized patients with a median of concentration increasing from 23,155 cp/mL in healthy donor to 103,482 cp/mL in critical cases.

Moreover, the correlation between death event and a plasmatic RNase P RNA concentration above 4.63 log cp/mL at hospital admission allowed the use of this quantitative plasmatic biomarker as a prognosis tool in COVID-19 hospitalized patients in complement to conventionally collected clinical parameters. These observations reflect the impressive clinical value of plasma RNase P RNA as a surrogate biomarker of COVID-19-induced global cell/tissue damage and the severity of COVID-19 pathology.

### REFERENCES

Chen L et al, Early Transmission Dynamics in Wuhan, China, of Novel Coronavirus-Infected Pneumonia. N. Engl. J. Med. 2020, 382, 1199-1207
Chen X, et al. Detectable serum SARS-CoV-2 viral load (RNAaemia) is closely correlated with drastically elevated interleukin 6 (IL-6) level in critically ill COVID-19 patients. Clin Infect Dis 2020; published online April 17. DOI:10.1093/cid/ciaa449.
Chen Y et al., Emerging coronaviruses: Genome structure, replication, and pathogenesis. J. Med. Virol. 2020; 92: 418-23.
Chen W et al, Emerging Microbes & Infections, 2020, VOL. 9, 469-473
Grasselli G et al, COVID-19 Lombardy ICU Network, Baseline Characteristics and Outcomes of 1591 Patients Infected With SARS-CoV-2 Admitted to ICUs of the Lombardy Region, Italy. JAMA, 2020, 323(16):1574-1581
Guan et al, China Medical Treatment Expert Group for Covid-19, Clinical Characteristics of Coronavirus Disease 2019 in China. N. Engl. J. Med, 2020; 382:1708-1720
Han et al. Prominent changes in blood coagulation of patients with SARS-CoV-2 infection. Clin Chem Lab Med 2020; published online March 16. DOI:10.1515/cclm-2020-0188.
Huang C et al. Clinical features of patients infected with 2019 novel coronavirus in Wuhan, China. Lancet 2020; 395: 497-506.
Li Q et al. Early transmission dynamics in Wuhan, China, of novel coronavirus-infected pneumonia. N Engl J Med 2020; 382:1199-207.
Pekin, et al, Quantitative and sensitive detection of rare mutations using droplet-based microfluidics. Lab on a chip (2011), 11(13): 2156-66.
Perkins, et al, Droplet-based digital PCR: application in cancer research. Advanced in clinical Chemistry (2017), 79: 43-91.
Ranieri et al, Acute respiratory distress syndrome: the Berlin Definition. JAMA. 2012; 307(23):2526-33
Taly, et al, Detecting Biomarkers with microdroplet technology. Trends in Molecular Medicine. 2012 July; 18(7):405-16.
Tang et al. Abnormal coagulation parameters are associated with poor prognosis in patients with novel coronavirus pneumonia. J Thromb Haemost 2020; 18: 844-7.
Yang F et al. Analysis of 92 deceased patients with COVID-19. J Med Virol 2020; published online April 15. DOI:10.1002/jmv.25891.
Yu et al. Quantitative Detection and Viral Load Analysis of SARS-CoV-2 in Infected Patients. Clin Infect Dis 2020; published online March 28. DOI:10.1093/cid/ciaa345.

1. A method for classifying a subject infected with SARS coronavirus, which method comprises measuring the quantity of circulating RNA of RNase P in a sample of the subject, wherein the sample is a blood, plasma or serum sample of the subject.

2. The method of claim 1, wherein the SARS coronavirus is SARS-CoV-2.

3. The method of claim 1, wherein the sample has been collected at DO and/or at different points of time between DO and D10, preferably at DO, D3, D6, D9, wherein DO (day 0) is the first day of symptoms of a SARS-induced disease or is the day wherein the subject has been diagnosed with a SARS coronavirus infection.

4. The method of claim 1, wherein the sample has been collected between 8 to 12 days after first symptoms of a SARS coronavirus infection.

5. The method of claim 1, wherein the subject shows hypoxemia.

6. The method of claim 1, wherein a quantity of circulating RNA of RNase P of 4.5 log₁₀ copies/mL or more is indicative of a severe case, or wherein a quantity of circulating RNA of RNase P of 5 log₁₀ copies/mL or more is indicative of a critical case.

7. A method for determining a risk for a subject to develop or show an aggravation of, a SARS coronavirus, preferably SARS-CoV-2, -induced acute pulmonary failure and/or systemic damage, which method comprises measuring the quantity of circulating RNA of RNase P in a sample of the subject, wherein the sample is a blood, plasma or serum sample of the subject, preferably wherein the subject shows hypoxemia.

8. The method of claim 7, wherein the sample has been collected between 8 to 12 days after first symptoms of a SARS coronavirus infection.

9. The method of claim 7, wherein the sample has been collected at DO and/or at different points of time between DO and D10, preferably at DO, D3, D6, D9, wherein DO (day 0) is the first day of symptoms of a SARS-induced disease or is the day wherein the subject has been diagnosed with a SARS coronavirus infection.

10. The method of claim 7, wherein the subject shows hypoxemia and the method is for use in assessing the risk of aggravation to a more severe stage of pulmonary failure and/or systemic damage.

11. The method of claim 7, wherein a quantity of circulating RNA of RNase P of 4.5 log₁₀ copies/mL or more is indicative of a subject likely develop an acute pulmonary failure, such as an acute respiratory distress syndrome (ARDS), and/or systemic damage, e.g. cardiovascular injury, renal injury, liver injury and/or multiple organ failure, or to die.

12. A method for monitoring efficacy of a therapeutic treatment against a SARS coronavirus infection or SARS coronavirus-induced acute pulmonary failure and/or systemic damage in a subject, which method comprises measuring the quantity of circulating RNA of RNase P in a sample of the subject, wherein the sample is a blood, plasma or serum sample of the subject collected at different points of time before, and during and/or after the subject has been subjected to the therapeutic treatment, preferably wherein the SARS coronavirus is SARS-CoV-2.

13. The method of claim 1, wherein the quantity of circulating RNA of RNase P is measured by a digital probe-based RT-PCR, preferably a digital droplet RT-PCR.

14. A method for determining whether a subject infected with SARS coronavirus, preferably SARS-CoV-2, is likely to die of the infection, which method comprises measuring the quantity of circulating RNA of RNase P in a sample of the subject, wherein the sample is a blood, plasma or serum sample of the subject, and a quantity of circulating RNA of RNase P of 4.5 log₁₀ copies/mL or more is indicative that the subject is at high risk of dying within three weeks.

15. The method of claim 14, wherein the sample has been collected at DO and/or at any day between D0 and D10, wherein D0 (day 0) is the first day of symptoms of a SARS-induced disease or is the day wherein the subject has been diagnosed with a SARS coronavirus infection, wherein a quantity of circulating RNA of RNase P of 4.5 log₁₀ copies/mL or more is indicative that the subject is at high risk of dying within one to three weeks from DO.

16. The method of claim 7, wherein the quantity of circulating RNA of RNase P is measured by a digital probe-based RT-PCR, preferably a digital droplet RT-PCR.

17. The method of claim 12, wherein the quantity of circulating RNA of RNase P is measured by a digital probe-based RT-PCR, preferably a digital droplet RT-PCR.
